(19) United States
(12) Patent Application Publication
(10) Pub. No.: US 2023/0229906 A1
(43) Pub. Date: Jul. 20, 2023

(54) ESTIMATING THE EFFECT OF AN ACTION USING A MACHINE LEARNING MODEL

(71) Applicant: Microsoft Technology Licensing, LLC, Redmond, WA (US)

(72) Inventors: Cheng ZHANG, Cambridge (GB); Javier ANTORAN, Cambridge (GB); Adam Evan FOSTER, Cambridge (GB); Maria DEFANTE, Redmond, WA (US); Steve THOMAS, Redmond, WA (US); Tomas GEFFNER, Amherst, MA (US); Miltiadis ALLAMANIS, Cambridge (GB); Karen FASSIO, Kirkland, WA (US); Daniel TRUAX, Redmond, WA (US)

(21) Appl. No.: 17/579,877

(22) Filed: Jan. 20, 2022

(51) Int. Cl. G06N 3/08; G06N 3/04

(52) U.S. Cl. CPC G06N 3/08; G06N 3/0454

(57) ABSTRACT

A computer-implemented method comprising: accessing a machine learning, ML, model that is operable to sample a causal graph from a graph distribution describing different possible graphs, wherein nodes represent the different variables of said set and edges represent causation, and the graph distribution comprises a matrix of probabilities of existence and causal direction of potential edges between pairs of nodes, and wherein the ML model is trained to be able to generate a respective simulated value of a selected variable from among said set based on the sampled causal graph. The method further comprises using the ML model to estimate a treatment effect from one or more intervened-on variables on another, target variable from among the variables of said set.

### BACKGROUND

Neural networks are used in the field of machine learning and artificial intelligence (AI). A neural network comprises plurality of nodes which are interconnected by links, sometimes referred to as edges. The input edges of one or more nodes form the input of the network as a whole, and the output edges of one or more other nodes form the output of the network as a whole, whilst the output edges of various nodes within the network form the input edges to other nodes. Each node represents a function of its input edge(s) weighted by a respective weight, the result being output(s) on its output edge(s). The weights can be gradually tuned based on a set of training data so as to tend towards a state where the output of the network will output a desired value for a given input.

Typically the nodes are arranged into layers with at least an input and an output layer. A “deep” neural network comprises one or more intermediate or “hidden” layers in between the input layer and the output layer. The neural network can take input data and propagate the input data through the layers of the network to generate output data. Certain nodes within the network perform operations on the data, and the result of those operations is passed to other nodes, and so on.

Each node is configured to generate an output by carrying out a function on the values input to that node. The inputs to one or more nodes form the input of the neural network, the outputs of some nodes form the inputs to other nodes, and the outputs of one or more nodes form the output of the network. At some or all of the nodes of the network, the input to that node is weighted by a respective weight. A weight may define the connectivity between a node in a given layer and the nodes in the next layer of the neural network. A weight can take the form of a scalar or a probabilistic distribution. When the weights are defined by a distribution, as in a Bayesian model, the neural network can be fully probabilistic and captures the concept of uncertainty. The values of the connections between nodes may also be modelled as distributions. The distributions may be represented in the form of a set of samples or a set of parameters parameterizing the distribution (e.g. the mean μ and standard deviation a or variance σ²).

The network learns by operating on data input at the input layer, and adjusting the weights applied by some or all of the nodes based on the input data. There are different learning approaches, but in general there is a forward propagation through the network from input layer to output layer, a calculation of an overall error, and a backward propagation of the error through the network from output layer to input layer. In the next cycle, each node takes into account the back propagated error and produces a revised set of weights. In this way, the network can be trained to perform its desired operation.

Training may employ a supervised approach based on a set of labelled training data. Other approaches are also possible, such as a reinforcement approach wherein the network each data point is not initially labelled. The learning algorithm begins by guessing the corresponding output for each point, and is then told whether it was correct, gradually tuning the weights with each such piece of feedback. Another example is an unsupervised approach where input data points are not labelled at all and the learning algorithm is instead left to infer its own structure in the experience data.

Other forms of machine learning model are also known, other than just neural networks, for example clustering algorithms, random decision forests, and support vector machines.

### SUMMARY

Some machine learning models can be designed to perform causal discovery using observational data or both observational and interventional data. That is, for a set of variables (e.g. [x₁, x₂,x₃]), the model when trained can estimate a likely causal graph describing the causal relationships between these variables. E.g. in the case of three variables a simple causal graph could be x₁→x₂→x₃, meaning that x₁ causes x₂ and x₂ causes x₃ (put another way, x₃ is an effect of x₂ and x₂ is an effect of x₁). Or as another example, x₁→x₂→x₃, means that x₁ and x₃ are both causes of x₂ (x₂ is an effect of x₁ and x₃). However, in the past such models have only be used for causal discovery alone, not for treatment effect estimation for decision making. Another type of model aims to do treatment effect estimation, which commonly assumes that the causal graph is already given by the user. Such methods do not currently work with unknown causal graphs. However it is appreciated herein that in many applications, users would benefit from the ability to perform treatment effect estimation for decision making with observational data only, without needing to know the causal graph.

The present disclosure provides an integrated machine learning model that both models the causal relationships between variables and performs treatment effect estimation.

According to one aspect disclosed herein, there is provided computer-implemented method comprising accessing a machine learning, ML, model that is operable to sample a causal graph from a graph distribution describing different possible graphs. In a causal graph, nodes represent the different variables of said set and edges represent causation, and the graph distribution comprises a matrix of probabilities of existence and causal direction of potential edges between pairs of nodes. The ML model has been pre-trained to be able to generate a respective simulated value of a selected variable from among said set based on the sampled causal graph. The method further comprises using the (trained) ML model to estimate a treatment effect from one or more intervened-on variables on another, target variable from among the variables of said set. This is done by: a) selecting the target variable as the selected variable to be simulated by the ML model; b) fixing the input value of each intervened-on variable to a specified value, including disregarding any edge directed from any parent of the intervened-on variable into the intervened-on variable in the sampled causal graph; c) sampling a causal graph from the graph distribution and observing the corresponding simulated value of the target variable; d) repeating c) multiple times, re-sampling the causal graph from the graph distribution each time; and e) determining an expectation of the target variable by averaging the simulated values of the target variable from c)-d) over the multiple sampled graphs, thus giving the estimated treatment effect.

By averaging over multiple possible causal graphs sampled from a distribution, this advantageously allows the method to exploit a model that has been trained for causal discovery in order to also estimate treatment effects. The method thus enables “end-to-end” causal inference.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. Nor is the claimed subject matter limited to implementations that solve any or all of the disadvantages noted herein.

### BRIEF DESCRIPTION OF THE DRAWINGS

To assist understanding of embodiments of the present disclosure and to show how such embodiments may be put into effect, reference is made, by way of example only, to the accompanying drawings in which:

FIG. 1 is a schematic block diagram of a system in accordance with embodiments disclosed herein,

FIG. 2 is a schematic computation diagram illustrating a machine learning model in accordance with the present disclosure,

FIG. 3 schematically illustrates an example of a causal graph,

FIG. 4 is schematic sketch of an example of a probabilistic distribution,

FIG. 5 schematically illustrates another example of a causal graph,

FIG. 6 is a schematic computation diagram illustrating a further machine learning model in accordance with embodiments disclosed herein,

FIG. 7 is a schematic flowchart of a method of training a model in accordance with the present disclosure, and

FIG. 8 is a schematic flowchart of a method of making treatment effect estimations using a trained model in accordance with embodiments disclosed herein.

### DETAILED DESCRIPTION OF EMBODIMENTS

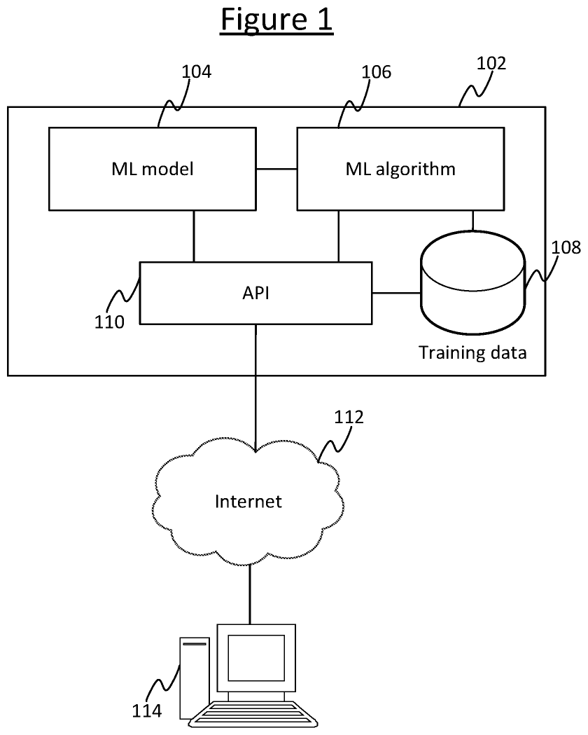

FIG. 1 illustrates an example system according to embodiments of the present disclosure. The system comprises a server system 102 of a first party, a network 112, and a client computer 114 of a second party. The server system 102 and client computer 114 are both operatively coupled to the network 112 so as to be able to communicate with one another via the network 112. The network 112 may take any suitable form and may comprise one or more constituent networks, e.g. a wide area network such as the Internet or a mobile cellular network, a local wired network such as an Ethernet network, or a local wireless network such as a Wi-Fi network, etc.

The server system 102 comprises processing apparatus comprising one or more processing units, and memory comprising one or more memory units. The, or each, processing unit may take any suitable form, e.g. a general purpose processor such as a CPU (central processing unit); or an accelerator processor or application specific processor such as a dedicated AI accelerator processor or a repurposed GPU (graphics processing unit), DSP (digital signal processor), or cryptoprocessor, etc. The, or each, memory unit may also take any suitable form, e.g. an EEPROM, SRAM, DRAM or solid state drive (SSD); a magnetic memory such as a magnetic disk or tape; or an optical medium such as an optical disk drive, quartz glass storage or magneto-optical memory; etc.

In the case of multiple processing units and/or memory units, these may be implemented in the same physical server unit, or different server units in the same rack or different racks, or in different racks in the same data centre or different data centres at different geographical sites. In the case of multiple server units, these may be networked together using any suitable networking technology such as a server fabric, an Ethernet network, or the Internet, etc. Distributed computing techniques are, in themselves, known in the art.

The memory of the server system 102 is arranged to store a machine learning (ML) model 104, a machine learning algorithm 106, training data 108, and an application programming interface (API) 110. The ML model 104, ML algorithm 106 and API 110 are arranged to run on the processing apparatus of the server system 102. The ML algorithm 106 is arranged so as, when run, to train the ML model 104 based on the training data 108. Once the model 104 is trained, the ML algorithm 106 may then estimate treatment effects based on the trained model. In some cases, after the ML model 104 been trained based on an initial portion of training data 108 and been made available for use in treatment effect estimation, training may also continue in an ongoing manner based on further training data 108, e.g. which may be obtained after the initial training.

The API 110, when run, allows the client computer 114 to submit a request for treatment effect estimation to the ML algorithm 106. The ML model 104 is a function of a plurality of variables. The request may specify a target variable to be examined, and may supply input values of one or more other variables (including intervened values and/or conditioned values). In response, the ML algorithm 106 may control the ML model 104 to generate samples of the target variable given the intervened and/or conditioned values of the one or more other variables. The API 110 returns the result of the requested causal query (the estimated treatment effect) to the client computer 114 via the network 112.

In embodiments, the API may also allow the client computer to submit some or all of the training data 108 for use in the training.

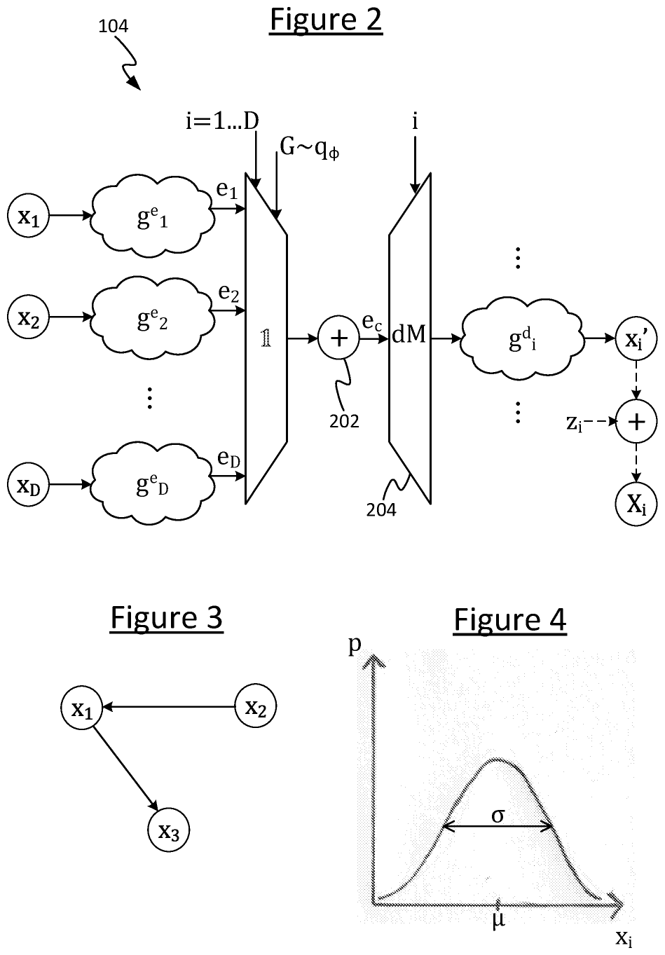

FIG. 2 schematically illustrates an example implementation of the machine learning model 104. The ML model 104 comprises a respective encoder gr and a respective decoder e for each of a plurality of variables xᵢ, where i is an index running from 1 to D where D>1.

Each variable represents a different property of a subject being modelled. The subject may for example be a real-life entity, such as a human or other living being; or a mechanical, electrical or electronic device or system, e.g. industrial machinery, a vehicle, a communication network, or a computing device etc.; or a piece of software such as a game, operating system software, communications software, networking software, or control software for controlling a vehicle or an industrial processor or machine.

For instance in a medical example, the variables represent different properties of a person or other living being (e.g. animal). One or more of the variables may represent a symptom experienced by the living being, e.g. whether the subject is exhibiting a certain condition such as a cough, sore throat, difficulty breathing, etc. (and perhaps a measure of degree of the condition), or a measured bodily quantity such as blood pressure, heart rate, vitamin D level, etc. One or more of the variables may represent environmental factors to which the subject is exposed, or behavioural factors of the subject, such as whether the subject lives in an area of high pollution (and perhaps a measure of the pollution level), or whether the subject is a smoker (and perhaps how many per day), etc. And/or, one or more of the variables may represent inherent properties of the subject such as a genetic factor.

In the example of a device, system or software, one or more of the variables may represent an output state of the device, system or software. One or more of the variables may represent an external factor to which the device, system or software is subjected, e.g. humidity, vibration, cosmic radiation, and/or a state of one or more input signals. And/or, one or more of the variables may represent an internal state of the device, system or software, e.g. an error signal, resource usage, etc.

One, more or all of the variables may be observed or observable. In some cases, one or more of the variables may be unobserved or unobservable.

Each respective encoder gᵢ^{e} is arranged to receive an input value of its respective variable xᵢ, and to generate a respective embedding eᵢ (i.e. a latent representation) based on the respective input value. As will be familiar to a person skilled in the art, an embedding, or latent representation or value, is in itself a known concept. It represents in the information in the respective input variable an abstracted form, typically in a compressed form, which is learned by the respective encoder during training. The embedding may be a scalar value, or may be a vector of dimension ‘embedding_dim’ which is greater than 1.

Note that a “value” as referred to herein could be a vector value or a scalar value. For example if one of the variables is an image (e.g. a scan of the subject), then the “value” of this vector variable is the array pixel values for the image.

The different variables xᵢ may have a certain causal relationship between them, which may be expressed as a causal graph. A causal graph may be described as comprising a plurality of nodes and edges (note that these are not the same thing as the nodes and edges mentioned earlier in the context of a neural network). Each node represents a respective one of the variables xᵢ in question. The edges are directional and represent causation. I.e. an edge from xᵢ₌ₖ to xᵢ₌ₗ represents that xₖ causes x₁ (x₁ is an effect of xₖ). A simple example involving three variables is shown in FIG. 3. In this example x₂ causes x₁, and x₁ causes x₃. For example x₃ may represent having a respiratory virus, x₁ may represent a lung condition and x₂ may represent a genetic predisposition.

Of course other possible graphs are possible, including those with more variables and other causal relationships.

A given graph G may be expressed as a matrix, with two binary elements for each possible pair of variables xᵢ₌ₖ, xᵢ₌ₗ; one binary element to represent whether an edge exists between the two variables, and one to represent the direction of the edge if it exists (e.g. 0=no edge from k to l, 1=edge from k to l, or vice versa). For example for three variables this could be written as:

$$G = ( \begin{matrix}Exists 1 , 2 & Dir 1 , 2 \\ Exists 1 , 3 & Dir 1 , 3 \\ Exists 2 , 3 & Dir 2 , 3\end{matrix} )$$

Other equivalent representations are possible. E.g. an alternative representation of the probabilities of existence and direction of edges in a graph would be:

$$G = ( \begin{matrix}Exists 1 \to 2 & Exists 1 \to 3 & Exists 2 \to 3 \\ Exists 2 \to 1 & Exists 3 \to 1 & Exists 3 \to 1\end{matrix} )$$

For any given situation, the actual causal graph may not be known. A distribution q_{ϕ} of possible graphs may be expressed in a similar format to G, but with each element comprising a parameter ϕ (phi, also drawn φ) representing a probability instead of a binary value.

$${q}_{φ} = ( \begin{matrix}φ\_exists1 , 2 & φ\_dir 1 , 2 \\ φ\_exists1 , 3 & φ\_dir1 , 3 \\ φ\_exists2 , 3 & φ\_dir2 , 3\end{matrix} )$$

(Or an equivalent representation.) In other words the parameter ϕ_exists1,2 represents the probability that an edge between x₁ and x₂ exists; ϕ_dir1,2 represents the probability that the direction of the possible edge between x₁ and x₂ is directed from x₁ to x₂ (or vice versa); parameter ϕ_exists1,2 represents the probability that an edge between x₁ and x₃ exists; etc.

Returning to FIG. 2, the ML model 104 further comprises a selector [symbol] , a combiner 202 and a demultiplexer 204, It will be appreciated that these are schematic representations of functional blocks implemented in software. The selector [symbol] is operable to sample a causal graph G from the distribution. This means selecting a particular graph G (with binary elements) whereby the existence and direction of the edges are determined pseudorandomly according to the corresponding probabilities in the distribution q_{ϕ}.

Preferably the possible graphs are constrained to being directed acyclic graphs (DAGs), for the sake of practicality and simplicity of modelling.

The selector [symbol] also receives a value of the index i for a selected target variable xᵢ. For the currently selected variable xᵢ (node of index i in the graph), the selector [symbol] selects the respective embeddings e_{Pa(i)} generated by the respective encoders g_{Pa(i)}^{e} of the parents Pa(i) of the node i (variable xᵢ) in the currently sampled graph G, and inputs these into the combiner 202.

The combiner 202 combines the selected embeddings e_{Pa(i)} into a combined embedding e_{c}. In embodiments the combination is a sum. In general a sum could be a positive or negative sum (a subtraction would be a sum with negative weights). The combined (summed) representation thus has the same dimension as a single embedding, e.g. ‘embedding_dim’. In alternative implementations however it is not excluded that another form of combination could be used, such as a concatenation.

The demultiplexer 204 also receives the index i of the currently selected variable xᵢ, and supplies the combined embedding e_{c} into the input of the decoder gᵢ^{d} associated with the currently selected variable xᵢ. This generates a value of a respective noiseless reconstructed version x′ᵢ of the respective variable xᵢ based on the combined embedding e_{c}.

The encoders g_{i=1 . . . D}^{e} and decoders g_{i=1 . . . D}^{d} are constituent machine learning models comprised by the overall ML model 104. They are each parameterized by respective sets of parameters θ which are tuned during learning. In embodiments, each of the encoders g_{i=1 . . . D}^{e} and decoders g_{i=1 . . . D}^{d} is a respective neural network (in which case their parameters may be referred to as weights). However it is not excluded that some or all of them could instead be implemented with another form of constituent machine learning model.

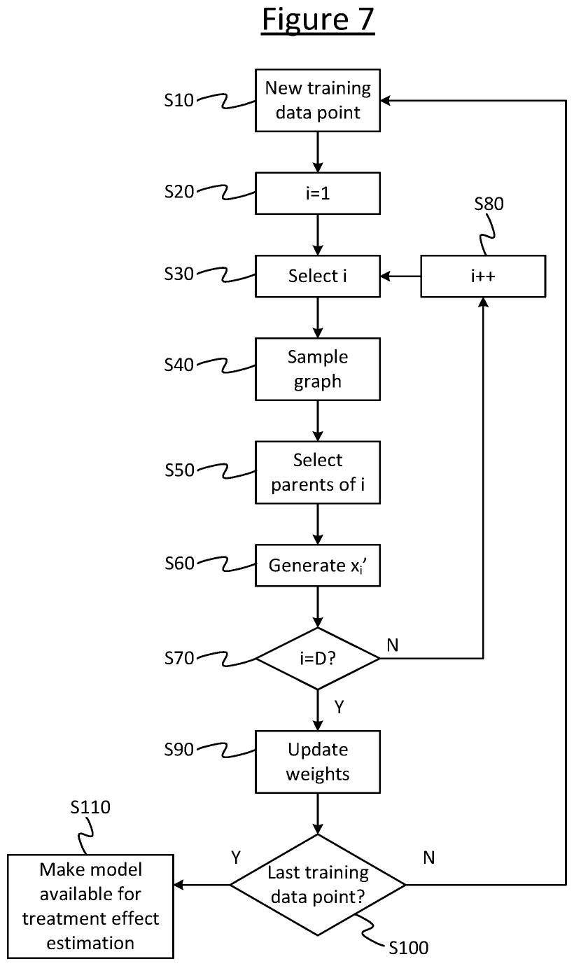

FIG. 7 schematically represents a method of training the model 104 based on a training data 108. The training data 108 comprises a plurality of training data points. Each data point [x₁ . . . x_{D}] comprises a set of input values, one respective value for each of the variables xᵢ.

The method processes each of the training data points, at least in an initial portion of the training data 108. At step S10 the method takes a new data point from memory, and at step S20 the index i is set to a first value (e.g. i=1) to specify a first target variable xᵢ from among the set of variables in the data point. At step S30, this causes the selector [symbol] to select the target variable xᵢ with the currently set value of the index i as the variable to be processed. At step S40 the selector [symbol] samples a random graph G from the distribution q_{ϕ}. At step S50, the selector [symbol] selects the parents Pa(i) of the target variable xᵢ (node i in the graph) and supplies the respective embeddings e_{Pa(i)} from the encoders g_{Pa(i)}^{e} of the selected parents into the combiner 202. At step S60, the combiner 202 combines (e.g. sums) the embeddings of the selected parents Pa(i) into the combined embedding e_{c}, and the demultiplexer 204 selects to supply the combined embedding e_{c} into the decoder gᵢ^{d} of the target variable xᵢ. The respective decoder gᵢ^{d} is thus caused to generate a noiseless reconstruction xᵢ′ of the selected target variable xᵢ.

This could equally be expressed as:

$${x}_{i}^{′} = {ℊ}_{i}^{d} ( {\sum }_{j ∈ Pa ( i )} {ℊ}_{j}^{e} ( {x}_{j} ) )$$

where Σj∈Pa(i) is another way of writing the operations of the selector [symbol] and combiner 202. Preferably, G is a DAG (directed and acyclic) so that it is valid to generate the value of any node in this way. The difference xᵢ−x′ᵢ may be referred to as the residual noise.

The process is repeated for each variable xᵢ in the set of variables, i.e. for i=1 . . . D, thus creating a full reconstructed set of values [x′₁ . . . x′_{D}] for the data point in question. This is represented schematically by steps S70 and S80 in FIG. 7, looping back to S30. However note that this the “loop” may be a somewhat schematic representation. In practice some or all of the different variables xᵢ for a given data point could be processed in parallel.

At step S90 the ML algorithm 106 applies a training function which updates the parameters (e.g. weights) θ of the encoders g_{i=1 . . . D}^{e} and decoders g_{i=1 . . . D}^{d}. Simultaneously it also updates the parameters ϕ of the distribution q_{ϕ} of possible graphs. The training function attempts to update the parameters θ, ϕ in such a way as to reduce a measure of overall difference between the set of input values of the set of input variables [x₁ . . . x_{D}] and the reconstructed version of the set variables [x′₁ . . . x′_{D}]. For example in embodiments the training function is an evidence lower bound (ELBO) function. Training techniques of this kind are, in themselves, known in the art, e.g. based on stochastic back propagation and gradient descent.

During the training phase, xᵢ is an observed data value that is attempted to be reconstructed. The residual xᵢ−x′ᵢ between the reconstruction and the observation may be used to compute the ELBO objective.

In embodiments, the model 104 may be a probabilistic model that it assigns a joint probability (likelihood) p (X₁, . . . ,X_{D}) to all the variables X₁, . . . ,X_{D}; where Xᵢ represents the noiseless reconstruction xᵢ combined with a random noise term zᵢ, e.g. additively (Xᵢ=x′ᵢ+zᵢ), and zi may be sampled from a random distribution, zᵢ^{˜}p(zᵢ). In such embodiments, then in order to train the model 104, one first collects a real-world data point with D-dimensional features (x_1, x_2, . . . , x_D), and then maximizes the likelihood of it being generated by the model. That is to say, the training operates to try to maximize p(X₁=x₁, X₂=x₂, . . . ,X_{D}=x_{D}). This is done by adjusting the parameters (e.g. weights) θ of the model 104. The ELBO mentioned previously is an example of a numerical approximation to the quantity p(X₁=x₁, X₂=x₂, . . . , X_{D}=x_{D}). ELBO is typically much easier to compute than p(X₁=x₁, X₂=x₂, . . . , X_{D}=x_{D}).

The above-described process is repeated for each of the data points in the training data 108 (or at least an initial portion of the training data). This is represented schematically in FIG. 7 by step S100 and the loop back to S10. Though again, this form of illustration may be somewhat schematized and in some implementations, batches of data points could be reconstructed in parallel, and the model parameters updated based on the batches.

At the beginning of the overall training method the graph distribution q_{ϕ} may start with some predetermined set of probabilities, e.g. all elements 0.5 for the existence and direction of each possible edge, or using some prior domain knowledge to inform which edges are possible or impossible, or more or less likely. Then, as the model is trained with more and more data points, the probabilities (i.e. the parameters ϕ of q_{ϕ}) are gradually learned in parallel with the parameters θ (e.g. weights) of the encoders and decoders q_{i=1 . . . D}^{e}, g_{i=1 . . . D}^{d}.

An instance of the above-described model and the method of training it is known in that after, in the form of a model known as “FCause”. However, in the past the FCause model has only ever been used for causal discovery, i.e. to estimate a causal graph. It has not been used to directly perform treatment effect estimation.

According to the present disclosure, once the model 104 has been trained sufficiently based on all the data points in the training data 108 (or at least an initial portion of the initial training data), then at step S110 the trained ML model 104 is made available to be used for treatment effect estimation (i.e., answering causal queries). In embodiments, this may comprise making the model 104 available to via the API 110 to estimate treatment effects/answer causal queries requested by the client computer 114.

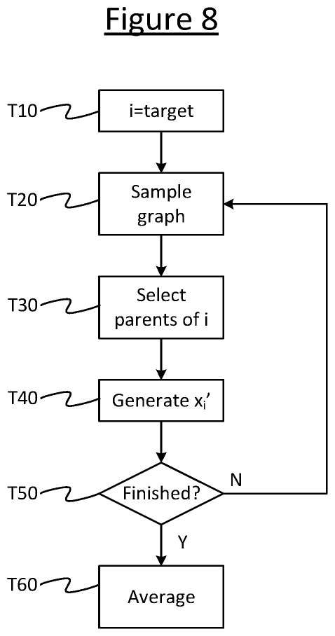

A method of using the ML model 104 to estimate a treatment effect is shown schematically in FIG. 8.

At step T10 the index i is set to that of a target variable xᵢ whose treatment effect is to be estimated. In addition, the input values of one or more “intervened-on” variables are also set to their known values. The intervened-on variables are variables whose values are set to some specified value, to represent that the property that they represent has been controlled (the treatment, i.e. an intervention on the modelled property). An “intervened-on” variable could also be referred to as a treated variable or controlled variable. For example, in the medical application, the intervened-on variable(s) may represent one or more interventions performed on the subject, and the target variable may represent a possible symptom or condition of the subject (e.g. the presence of a certain disease). Or in the case where the subject is a device or software, the intervened-on variable(s) may represent one or more states that are set to defined values, and the target variable may represent a condition or state of the device or software that is being diagnosed.

At step T20, the selector [symbol] samples a graph G pseudorandomly from the distribution q_{ϕ} according to the probabilities ϕ. In other words the chance of there existing an edge in given direction between each pair of nodes i=k, i=1 in the sampled graph G is determined by the pair of parameters ϕ_exists(k,l); ϕ_dir(k, l). Although optionally, some of the learned probabilities could be overridden to predetermined values based on prior knowledge (e.g. in some scenarios a given causality could be ruled out—probability set to zero—or may be known to be unlikely, either based on a priori or empirical knowledge).

At step T30 the selector [symbol] selects the parents Pa(i) of the target variable xᵢ in the sampled graph G, and supplies the respective embeddings e_{Pa(i)} from the encoders g_{Pa(i)}^{e} of the selected parents into the combiner 202 to be combined (e.g. summed) into the combined embedding e_{c}. The demultiplexer 204 selects to pass the combined embedding e_{c} into the decoder gᵢ^{d} of the target variable xᵢ, thus causing it to generate a noiseless reconstruction of x′ᵢ.

Thus the selection [symbol] selects the parents of node i in the graph G, so it depends on both i and G. The index is set to a particular target node i that one is trying to estimate (as well as selecting a graph G from q_{ϕ}), and the model 104 outputs a noiseless reconstruction of xᵢ, denoted by x′ᵢ. During training, such reconstruction was formed of each node given the actual data for its parent nodes.

Now at test time, the model 104 is used in a slightly different way for treatment effect estimation. Here, the target variable xᵢ which is now treated as unknown, and the goal is to generate simulated values of the target variable, given other interventional variables. In some embodiments the simulated value could just be taken as the noiseless reconstructed value xᵢ. However for some purposes, the noiseless reconstruction x′ᵢ of the target variable xᵢ may not be considered preferable, and instead the full interventional distribution may be taken into account. This can characterized by the following sources of uncertainties: 1), different realizations of the graph G that could happen to have been selected during sampling (even an unlikely one), which can be modelled by simulating different graphs from q_{ϕ}; and 2), the randomness of the residual noise random variables zᵢ. The latter can be taken into account by the following simulation equation:

Xᵢ=gᵢ^{d}(Σ_{j∈Pa(i)}gⱼ^{e}(xⱼ))+zᵢ,zᵢ˜p(zᵢ).

In other words, during the treatment effect estimation phase, Xᵢ is a simulated value that is obtained through random sampling of zᵢ, wherein Xᵢ=x′ᵢ+zᵢ (except for x_{i=T}, the intervened-on variable). More generally in other possible implementations, the noise zᵢ could be combined with the noiseless reconstruction x′ᵢ in other ways in order to obtain Xi, not necessarily additively.

In the following description, reference may be made to x′ᵢ as the simulated value of target variable xᵢ. However generally the simulated value could simply be the noiseless reconstructed value x′ᵢ, or Xᵢ (which takes into account noise, e.g. Xᵢ=x′ᵢ+zᵢ), or a value which is simulated based on the reconstructed value x′ᵢ in any other way.

By whatever means the simulated value is determined, according to the present disclosure, an average is taken over multiple sampled graphs. In embodiments, an average is taken over multiple sampled graphs and residual noise variables zᵢ. In other words, for a given target variable xᵢ to be estimated, multiple values of variable Xᵢ are simulated based on different respective sampled graphs G, each time sampled randomly from both q_{ϕ} and zᵢ

This is represented schematically in FIG. 8 by the loop from step T50 back to T20, to represent that multiple simulated values of xi are determined (for a given target variable xᵢ), each determined based on a different randomly sampled graph G (each time sampled from the distribution q_{ϕ}) and optionally noises zᵢ. Again the illustration as a loop may be somewhat schematized, and in embodiments some or all of the different simulated values for a given target variable xᵢ, based on the different sampled graphs and sampled residual noises z, may in fact be computed in parallel.

In embodiments the average could be taken as a simple mean, median or mode of the different values of the simulated values x′ᵢ or X′ᵢ of the variable xᵢ (as simulated with the different sampled graphs and optionally residual noises). In embodiments, such averaging is based on estimating an expectation of the probabilistic distribution of the simulated values.

FIG. 4 schematically illustrates the idea of a probabilistic distribution p(xᵢ). The horizontal axis represents the value of the target variable being simulated, xᵢ, and the vertical axis represents the probability that the variable takes that value. If the distribution is modelled as having a predetermined form, it may be described by one or more parameters, e.g. a mean μ and standard deviation a or variance σ² in the case of a Gaussian. Other more complex distributions are also possible, which may be parameterized by more than two parameters, e.g. a spline function. As a convenient shorthand the probabilistic distribution may be referred to as a function the target variable of xᵢ, i.e. p(xi), in the sense that it models the distribution of the target variable xᵢ, but in fact it will be appreciated that the distribution is in fact determined based on the corresponding simulated values x′ᵢ or Xᵢ.

Based on the trained model 104, it is possible using statistical methods to estimate an expectation E[p(x_{Y})|do(x_{T}=val₁)] of the probabilistic distribution p of a particular target variable x_{i=Y} given the intervened value val1 of another, treatment/intervention variable x_{i=T}. In embodiments, the target variable x_{Y} may model an outcome of a treatment modelled by x_{T}. Note that “treatment” as used most broadly herein does not necessarily limit to a medical treatment or a treatment of a living being, though those are certainly possible use cases. In other examples, the treatment may comprise applying a signal, repair, debugging action or upgrade to an electronic, electrical or mechanical device or system, or software, where the effect may be some state of the device, system or software which is to be improved by the system. In embodiments, the actual real-world treatment may be applied in dependence on the estimation (e.g. expectation) of the effect of the modelled treatment, for example on condition that the treatment estimation (e.g. expectation) is above or below a specified threshold or within a specified range.

One suitable measure of expectation may be referred to as the average treatment effect (ATE). This may be expressed as:

E[p(x_{Y})|do(x_{T}=val1)]−E[p(x_{i=Y})|(do(x_{T}=val2)]

or

E[p(x_{Y})|do(x_{T}=val1)]−E[p(x_{i=Y})]

which could also be expressed E[p(x_{Y})|do(x_{T}=val1)]−E[p(x_{i=Y})|do(x_{T}=mean(x_{T}))], where val1 is some treatment, val2 is some other treatment, and “do” represents applying the treatment. In other words, the ATE is the difference between: a) the expectation of the distribution of the effect x_{Y} given the value val1 of a treatment x_{T} and b) the expectation of the distribution of the effect x_{Y} given the value val2 of a treatment x_{T}, or the difference between a) the expectation of the distribution of the effect x_{Y} given the value val1 of a treatment x_{T} and b) the expectation of the distribution of the effect x_{Y} without applying a value of the treatment x_{T}. Note that in variants the equality x_{T}=val1 or x_{T}=val2 could be replaced with another form of expression such as a “greater than”, “less than” or range type expression.

An estimation of E[p(x_{Y})═do(x_{T}=treatment) may be determined based on the modelled distributions of noise, together with the learned graph and arrow functions. This may be done using the noise distribution at node x_{Y}, and using noise samples from other upstream variables, plus knowledge of the graph and its behaviour under the intervention do(x_{T}=treatment), in order to thus make the ATE estimation calculation. The expectation E[p(x_{Y})|do(x_{T}=val)] of a target variable xv given a treatment x_{T}=val may be computed as:

E[g_{Y}^{d}(Σ_{i∈Pa(Y)}eᵢ)+z_{Y}]

where z_{Y} is a noise term. E is an average over everything that is random in the equation. Consider for example a simple graph:

x₁→x₂←x₃

where x₁ is the treatment x_{T}, and x₂ is the target variable x_{Y}. In the computation of E, x₁ is set to its known value. E may then be written:

E[d_{Y}^{d}(g₁^{e}(x₁=val)+g₃^{e}(x₃))+z_{Y}]

To compute E, one example method is simply to take the mean of the different sampled values x_{Y}′ or X_{Y} of the target variable xv, based on the different sampled graphs, optionally also including some random noise (e.g. additive noise) in each sample. Another option is to fit the sampled values of xv to a predetermined form of distribution, such shown as in FIG. 4, e.g. a Gaussian, normal or spline function (again optionally also including random noise). The average may then be determined as the average of the fitted distribution.

Note that during the determination of the multiple instances of x′ᵢ it is possible, especially for some distributions, that the same graph G ends up being sampled multiple times, or even every time. However the graph is still being freshly sampled each time and even if the sampled graphs turn out to be the same, they may still be described as different instances of the sampled graph, in the sense that they are individually sampled anew each time. In the case where the multiple graphs happen to be the same, then the averaging only averages the noise z.

In some cases, if the treated (i.e. controlled) variable has one or more parents, the graph G may be mutilated: G->G_{do(xT=val)}. E.g. consider the graph:

x₄→x₁→x₂←x₃

where again x₁ is the treatment x_{T}, and x₂ is the target variable x_{Y}. In the computation of E, x₁ is set to its known value. Therefore x₄ has no effect on the outcome of x₂. So in the determination of the expectation, the graph is mutilated to remove the node x₄ and the edge from x₄→x₁. In other words, fixing the value of the known (controlled) variable x₁ means that any effect of the edge from the parent of the known variable x₁.

Another type of average which may be determined according to embodiments disclosed herein may be referred to as the conditional ATE (CATE). This is the difference between: a) the expectation of the target variable xv given the treatment x_{T}=val1. conditional on an observation γ of at least one other of the variables x_{C} in the set, and b) the expectation of the target variable x_{Y} without the treatment (either with a different treatment val2 or no treatment) but still conditional on the same observation of x_{C}. That is,

E[p(x_{Y})|do(x_{T}=val1),x_{C}=γ]−E[p(x_{Y})|do(x_{T}=val2),x_{C}=γ]

or:

E[p(x_{Y})|do(x_{T}=val1),x_{C}=γ]−E[_{P}(x_{Y}),x_{C}=γ]

which could also be expressed E[p(x_{Y})|do(x_{T}=val1), x_{C}=γ]−E[p(x_{i=Y})|do(x_{T}=mean(x_{T})) x_{C}=γ]

Note that in variants the equality x_{T}=val1, x_{T}=val2, or x_{C}=γ could be replaced with another form of expression such as a “greater than”, “less than” or range type expression.

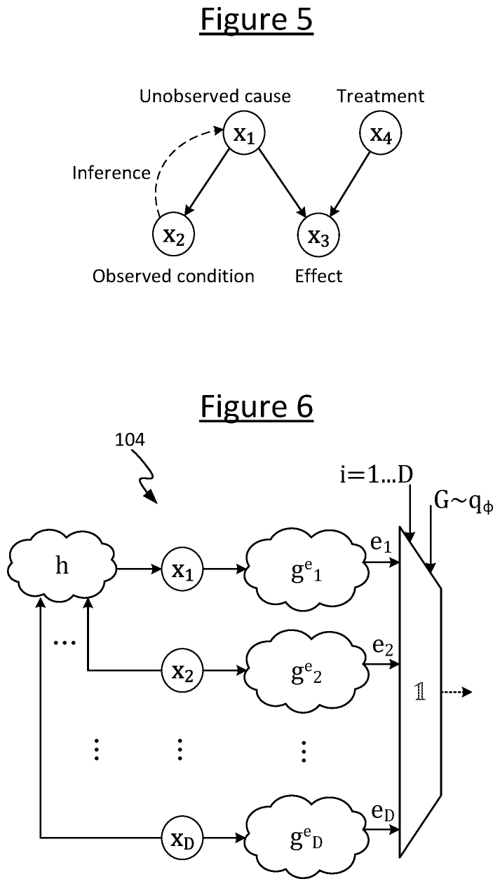

FIG. 5 illustrates by way of example why estimating the conditional treatment effect is not necessarily straightforward. In this example x₃ is the target x_{Y} whose treatment effect will be estimated and x₄ is the treatment x_{T}, where the treatment is the cause of the target effect. In addition, there is another, unobserved cause x₁ of the target effect, and another observable effect x₂ of the unobserved cause x₁. The variable x₂ is to be the observed condition x_{C}. For instance, in a medical example the target effect x₃ could be some condition or symptom of the subject (e.g. a respiratory problem), the treatment x₄ could be a possible medical intervention (e.g. taking some drug or vitamin), the unobserved cause x₁ may be a genetic factor, and the other observable cause x₂ may be some observable physical quality of the subject's body (e.g. body mass index). In general the unobserved cause could be unobservable, or merely unobserved.

The observable condition x₂ contains information about the unobserved cause x₁, which in turn can reveal information about the desired effect x₃ (=x_{Y}). For example if it is known that an athlete is in the Olympics and that they are a footballer, this reduces the probability that they are also a rower. In fact for any two variables that are effects of a common cause and whose intersection is not 100%, knowing something about one gives information about the other. E.g. if it is known that a subject has lung cancer and that they were exposed to a carcinogenic chemical other than tobacco, this reduces the probability that they were are smoker.

However the causal direction is from x₁→x₂, and the model 104 of FIG. 2 is only configured to learn effects of causes in the direction from cause to effect—it is not configured to learn inferences of effect from cause. I.e. it is not configured to “go against the arrows” in the figure (the directional causal edges).

To address this, as illustrated in FIG. 6, in embodiments the ML model 104 may be adapted to include at least one inference network h disposed between at least one observable condition x_{c} (x₂ in the example) and at least one unobservable potential cause (x₁ in the example) of the condition x_{c}. As illustrated in FIG. 6, in some such embodiments, the inference network h (or individual such networks) may be disposed between the unobserved cause and multiple potential effects (up to all the other variables). This will allow the model to learn which variable(s) may be an effect of the unobserved cause, if relationship is not prior knowledge.

The inference network(s) h may be trained at the training stage simultaneously along with the encoders g^{e} and decoders g^{d} and the parameters of the graph distribution q_{ϕ}, or alternatively after the rest of the model (see below). In embodiments the inference network h may comprise a neural network, in which case training the inference network comprises tuning the weights of the inference network. Alternatively the use of other forms of machine learning is not excluded for the inference network.

The inclusion of the inference model makes it possible to estimate a conditional expectation such as E[x_{Y}|do(x_{T}=val1), x_{C}=γ].

To estimate the conditional expectation, then initially during the estimation of the target variable x_{Y}, the conditional variable x_{c} is not fixed. In other words, the method proceeds as described above with respect to ATE, to obtain multiple different samples of x_{Y} based on multiple respective sampled graphs. At the same time, respective simulated samples x_{c} of the conditional variable are also obtained in the same way based on the respective sampled graphs. This gives a two dimensional set of samples (x_{Y}, x_{c}), which could be described as a plot or “scatter plot”. Then a predetermined form of function is fitted to the 2D set of samples (x_{Y}, x_{c}), such as a straight line, a curve, or a probabilistic distribution. After that, xc is set to its observed value in the fitted function, and a corresponding value of x_{Y} is read out from the fitted function. This is taken as the conditional expectation of x_{Y} given x_{c}.

At least two alternative variants to computing CATE may be employed, depending on implementation.

Variant I: estimate CATE using the same approach as used to estimate ATE, but performing a re-weighing of the terms inside of the expectations such that the condition (that gives CATE its name) is satisfied. This type of approach is known as an importance sampling technique. The weights of the different samples used to compute the expectation are provided by an inference network, which is trained together with the rest of the ML model 104.

Variant II: after the model 104 of FIG. 2 has been trained and a specific CATE query is received (e.g. via the API 110), the inference network h is trained to estimate the effect variable from the conditioning variable. To train this model, data simulated from the trained model of FIG. 2 is used, while applying some treatment specified in the query. Then the conditional average treatment effect is estimated by inputting the relevant value of the conditioning variable into the inference network h. It returns a distribution over effects from which the expected effect can be computed.

The main difference between the two approaches is that the first solely uses components learnt from the observed data during model training, while the second requires learning a new network after the model 104 has been trained. The reasoning for proposing both methods is that there are specifics settings where one or the other are more computationally efficient.

Note that the disclosed methods are not limited to the controlled (i.e. treated) variable x_{T} being a direct parent of the target variable x_{Y} (i.e. the variable whose treatment effect being estimated). The treated variable or the effect variable can be any arbitrary variable in the set x_{i=1 . . . D}, e.g. a grandparent of the target variable x_{Y}.

In embodiments, the simulation of the target variable takes into account a potential effect of all causal variables across the sampled graph. An example implementation of this is as follows. This may be used in conjunction with any of the ways of averaging discussed above, or others.

The method of estimating the target variable x_{Y} (e.g. the treatment effect) may comprise an inner and an outer loop. In the inner loop, the method loops through i=1 . . . D, so as to generate the simulated value xᵢ of each of the input variables xᵢ (even those that are not the target variable), except skipping over the one or more controlled (treated or known) variables x_{T} which are set to their known, fixed values (any edges into those nodes have been “mutilated” away, i.e. removed from the sampled graph). Then, proceeding to the next round (iteration) of the outer loop, the simulated values xᵢ of the non-controlled variables are fed back to the respective inputs of the model 104. In other words (other than for the one or more controlled variables x_{T}), the simulated values from the previous round or cycle (iteration) of the outer loop become the input values xᵢ of the current iteration of the outer loop to generate an updated set of values for the simulated variables xi. This may be repeated one or more further times, and the simulated values will start to converge (i.e. the difference between the input layer and output layer of the model 104 will get smaller each time). If noise is included the noise is frozen throughout a given inner loop, then re-sampled each outer loop. The total number of iterations of the outer loop may be predetermined, or the outer loop may be iterated until some convergence criterion is met. In embodiments the outer loop is iterated at least D−1 times, which guarantees convergence without needing to evaluate a convergence criterion.

This method advantageously allows causal effects to propagate throughout the graph. For example if x₁ causes x₂ and x₂ causes x₃, and an intervention is performed on x1 then the outer loop will be run at least two times to propagate the effect through to x3.

However the method of using an inner and outer loop is not essential. An alternative would be to perform a topological sort of the nodes and propagate effects through in a hierarchical fashion starting from the greatest grandparents or “source nodes” (those which are only causes and not effects). Or as another alternative, though less preferred, it is not necessary to propagate effects through multiple generations and instead only the effects of immediate parents of the target variable may be taken into account.

Further details of some example implementations of the various concepts discussed above are now described, by way of example only.

### Example Implementation

Questions of a causal nature are abundant in many fields in which machine learning is applied, including economics, medicine, biology, and fair machine learning. In business decision making, for example, practitioners may wish to forecast the effect of offering a promotion to certain customers on key indicators such as future revenue. To do so, it is not enough to look at correlations in historic data, instead we must estimate the causal effect of the promotion upon the downstream variables of interest.

Practitioners are therefore presented with the end-to-end causal inference problem of going from data to the estimation of causal quantities, such as the effect of a treatment without having full a priori knowledge of the causal relationships between variables. This end-to-end pipeline consumes observations of the relevant variables in observational (and possibly interventional) environments, and outputs estimates of average treatment effect (ATE) and conditional ATE (CATE). Unfortunately, most causal machine learning methods are not designed for this end-to-end problem, instead they draw a sharp distinction between causal discovery and causal inference.

In the structural equation modelling (SEM) framework, causal discovery refers to uncovering the directed acyclic graph (DAG) that models causal relationships between variables, whilst causal inference uses this DAG to perform calculations, yielding estimates of (C)ATE. Standard causal inference methods assume that the DAG is already known. The classical approach, then, is to use a causal discovery method to learn the DAG (or its Markov equivalence class), and then plug this into existing methods for causal inference.

Unfortunately, the assumptions required for the causal discovery algorithm are often mismatched with the assumptions of the latter causal inference. To move directly from data to causal inference in a seamless end-to-end pipeline would markedly accelerate many real-world uses of causal inference. Yet the challenges to making this a reality are significant.

Given only observational data, the true causal DAG may not be identifiable, meaning that different DAGs might fit the training data equally well. How do we deal with uncertainty in the DAG when estimating treatment effects? Furthermore, for a single DAG, there remains the problem of choosing a suitably flexible functional form as a model for the dependence of each child node on its parents. Learning both the DAG structure and the functional form of the data generating process involves simultaneous inference of discrete and continuous random variables, this presents a challenge for many machine learning algorithms that are tailored to continuous parameters.

The present disclosure provides a new framework for end-to-end causal inference that offers a practical and flexible method for moving directly from data to (C)ATE estimation, and so to real-world decision making. There is provided both a general framework that consumes any model that consists of both a distribution over possible graph structures, and fitted arrow functions (the functions that map the set of parents to a distribution over the child), and estimates (C)ATE.

There is also provided a specific flow-based model that satisfies these two requirements.

In embodiments the disclosed model provides a single, unified set of assumptions on the causal model, which are then used for jointly for discovery and inference. The model itself uses flows to model complex nonlinear arrow functions, as well as non-Gaussian noise. Ideas are used from continuous optimization-based causal discovery to learn a posterior distribution over DAGs. The model supports discrete and continuous variables, missing data, and partially specified prior knowledge on the graph, resulting in arguably the most complete model for learning causal structures from real world data. Once this powerful model has been trained, it can then be used in our general framework for (C)ATE estimation.

To address the identifiability challenges, a pragmatic approach is taken that seeks to make best use of the assumptions and the data that is available. The partial specification

of prior information is allowed for the DAG, allowing unknown features of the graph to be learned from data. Training is also allowed with both observational and interventional data. Finally, we marginalise over uncertainty in the DAG in our (C)ATE estimates.

Contributions that may be achieved by embodiments disclosed herein may be summarised as follows.

Firstly, a general framework for end-to-end causal inference. A Bayesian perspective is taken, that places a posterior distribution over possible DAGs. In treatment effect estimates, the method marginalises over the posterior uncertainty in the DAG.

Secondly, a specific flow-based model for causal modelling. The disclosed model incorporates several novel features that are useful in practical causal problems:
it allows the partial specification of prior information for the DAG, allowing unknown features of the graph to be learned from data,
its allows training with both observational and interventional data,
is uses a flow-based model that can treat a mixture of continuous and discrete data,
it models non-Gaussian additive noise for continuous variables.

### Preliminaries

Let x=(x₁, . . . , x_{D}) be a collection of random variables. Structural equation models (SEM) are a broadly accepted framework for describing causal relationships between the xᵢ. Unlike conventional models which describe the joint distribution p(x), a SEM provides a joint distribution for x and describes how this joint distribution should change under different interventions. Given a directed acyclic graph (DAG) G∈G on nodes {1, . . . ,D}, the observational joint distribution consists of a series of D structural equations of the form:

xᵢ=Fᵢ(x_{pa(i;G)},εᵢ) (1)

where Fᵢ is the ith ‘arrow function’, pa(i;G) is the set of parents of i in G, and εᵢ is a noise random variable that is independent of all other variables in the model. The following examples use additive noise SEMs, in which:

Fᵢ(x_{pa(i;G)}),εᵢ)=fᵢ(x_{pa(i;G)})+εᵢ. (2)

Under the interventional distribution in which a subset of nodes x_{T} (where T⊆{1, . . . , D}) is set to some fixed value a, the SEM simply replaces the structural equations for the i∈T with:

x₁=aᵢ if i∈T (3)

and the other structural equations are left unchanged. This distribution is denoted p(x|do(x_{T}=a)).

The SEM allows computation of the average treatment effect (ATE) of x_{T} on targets x_{Y} as:

$$A T E ( a , b ) = {p ( {x}_{Y} \vert do ( {x}_{T} = a ) )}_{} [ {x}_{Y} ] - {p ( {x}_{Y} \vert do ( {x}_{T} = b ) )}_{} [ {x}_{Y} ] \qquad ( 4 )$$

and the average treatment effect of x_{T} on xv conditional on x_{C}:

$$CATE ( a , b , c ) = {p ( {x}_{Y} \vert do ( {x}_{T} = a ) , {x}_{C} = c )}_{} [ {x}_{Y} ] - {p ( {x}_{Y} \vert do ( {x}_{T} = b ) , {x}_{C} = c )}_{} [ {x}_{Y} ] . \qquad ( 5 )$$

### Continuous DAG Characterisation

Let W∈RD×RD be a weighted adjancency matrix. It can be shown that:

h(W):=tr(exp(W⊙W))−D (6)

is nonnegative and equals 0 if and only if W corresponds to a DAG. Thus a DAG can be trained by augmented Lagrangian methods.

### FCause Revisited

FCause is an additive Gaussian SEM model:

$${x}_{i} = {ℊ}^{d} ( {\sum }_{j ∈ P a ( i ; G )} {G}_{ji} {ℊ}^{e} ( {x}_{j} , j ; {θ}_{e} ) , i ; {θ}_{d} ) + {z}_{i} ; {z}_{i} \sim ( 0 , {σ}_{i}^{2} I ) \qquad ( 7 )$$

in which the relationships among all variables in the graph fᵢ∀i∈T are modelled with an encoder-decoder neural network pair g^{e}: [symbol] → [symbol] and g^{d}: [symbol] → [symbol] , where dᵢ represents the dimensionality of variable i and dₑ is an embedding dimension. The noise variance σ² i is learnable, making FCause robust to re-scaling of the data. FCause places a factorised Bernoulli prior p(G) over graph edges and their directions. It supports DAGs:

$$p ( G ) ∝ ( h ( G ) = 0 ) {\prod }_{i , j} Bern ( {G}_{ij} ) \qquad ( 8 )$$

The likelihood for this model is intractable. Instead, the SEM parameters θ=(θ_{g}, θ_{f}, σ) and a variational distribution over graphs q_{φ}(G), which is chosen to be of the same family as the prior, are jointly optimised using an ELBO:

$$log {\prod }_{n} {p}_{0} ( {x}^{n} ) \geq {{q}_{ϕ} ( G )}_{} [ log \frac{p ( G ) {\prod }_{n} {p}_{θ} ( {x}^{n} \vert G )}{{q}_{ϕ} ( G )} ] . \qquad ( 9 )$$

Finally, FCause supports learning from partial observations. Let us denote the set of indices of observed variables as O, leaving U=\O for the unobserved. It is possible to approximately marginalise xU by introducing an imputation distribution q_{ψ}(x_{U}|x_{O}). This results in the following ELBO:

$${{q}_{ϕ} ( G ) , {q}_{ψ} ( {x}_{U} \vert {x}_{U} )}_{} [ log ( \frac{p ( G )}{{q}_{ϕ} ( G )} {\prod }_{n} \frac{{p}_{θ} ( {x}_{O}^{n} , {x}_{U} \vert G )}{{q}_{ψ} ( {x}_{U} \vert {x}_{O}^{n} )} ) ] . \qquad ( 10 )$$

### An End-to-End Causal Inference Pipeline

Taking an end-user's perspective, we seek to combine approaches to discovering causal relationships among variables with methods for causality-aware prediction or decision making into a single tool. To this end, there is presented herein an end-to-end (E2E) framework for causal inference. This framework gives the graph G a probabilistic treatment. In real-world settings, the causal relationships among variables can be very complicated. Thus, combining the output of causal discovery methods, that act on observations, with domain knowledge might not be enough to fully determine G. Instead embodiments herein model the uncertainty over the causal relationships that govern the data using a posterior over graphs:

$$p ( G \vert X ) = \frac{p ( X \vert G ) p ( G )}{{\sum }_{G} p ( X \vert G ) p ( G )} . \qquad ( 11 )$$

The likelihood tells about the degree of compatibility of a certain graph architecture with the observed data. For score-based discovery methods, the score may be taken to be log p(X|G). For functional discovery methods, the exogenous variable log-density may be used. Constraint-based methods can also be cast in this light by assuming a uniform distribution over all graphs in their outputted equivalence class G: log p(X|G)=−log|G|, ∀G ∈G. To what degree these methods succeed at constraining the space of possible graphs will depend on how well their respective assumptions are met and the amount of data available. Constructing a likelihood from purely-interventional or mixed data, as opposed to from purely observational data, can improve the sample-efficiency of graph inference.

The prior, p(G) reflects beliefs about the causal graph drawn from domain expertise. This probabilistic formation allows hard constraints about specific sets of edges which may be present, or soft beliefs about roughly how many edges should be active or which groups of edges are likely to appear together. A more informative prior drives inferences closer to the purely causal domain. The graph posterior (eq. 11) may be leveraged to introduce a new type of hybrid causal-probabilistic inference, which combines causal beliefs with probabilistic marginalisation over the parts of the graph not specified by these beliefs. The interventional distribution is given by:

[symbol]

_{p(G|X)}[p(x_{Y}|do(x_{T}=a),G′)], (12)

and treatment effect estimators by:

[symbol]

_{p(G|X)}[CATE(a,b,c,G)]. (13)

The disclosed framework can be understood as a probabilistic relaxation of traditional causal quantity estimators. It becomes equivalent to traditional causal inference when we are certain about the form of the graph p(G|X)=δ(G−Gᵢ).

### Fcause+, a Dgm for E2E Causal Inference

The E2E causal inference framework may be implemented using a single model approach based on FCause. In the case of FCause, the Bayesian update in Eq. (11) is performed implicitly, through variational expectation maximisation (E.q. 10). This results in the method directly outputting an approximate posterior over graphs q(G)≈p(G|X). Although originally conceived as a causal discovery method, FCause learns functional relationships among variable pairs connected by graph edges during its training. As a result, we can sample from the learnt E2E model p_{θ}(x) by first sampling a graph from the learnt posterior Gₘ˜q_{φ}(G) and then recursively applying the SEM equation section 3.3 in the order of the topologically sorted variables according to GK. For a given graph, the density of some observation vector a is computed by evaluating the base distribution density after inverting the SEM:

$${p}_{θ} ( x = a ) = {\prod }_{i} p ( {z}_{i} = ( {a}_{i} - {f}_{i} ( {a}_{Pa ( i ; {G}^{m} )} ) ) ) \qquad ( 14 )$$

noting that the transformation Jacobian is the identity (citation). Then the graphs are marginalised the graphs using montecarlo:

$${p}_{θ} ( x = a \vert {G}^{k} ) = \frac{1}{M} {\sum }_{m}^{M} {p}_{θ} ( x = a \vert {G}^{m} ) ; \qquad ( 15 )$$

$${G}^{m} \sim {q}_{ϕ} ( G ) .$$

In the rest of this section, methods are derived that allow using FCause to estimate causal quantities and propose a series of improvements, which relax FCause's assumptions and make it applicable to different types of data.

### Treatment Effect Computation

First it is described how to obtain inconditional interventional distributions from FCause in a simple manner. Then conditioning is introduced, which will involve more complex approximate inference machinery.

Treatment distribution density and ATE computation: Applying an intervention do(xT=b) can be understood as a modification to the causal graph resulting in the distribution:

p(x_{\T}|do(x_{T}=b),G)=p(x_{\T}|x_{T}=b,G_{do(x}_{_{T}}₎) (16)

where G_{do(x}_{_{T}}₎ has entries Gⱼᵢ=0, ∀j∈Pa(i), i∈T. Under G_{do(x}_{_{T}}₎, i∈T correspond to parent nodes and we have the following factorisation: p(x|G_{do(x}_{_{T}}₎)=p(x_{\T}|G_{do(x}_{_{T}}₎)Π_{i∈T}p(xᵢ). One can then evaluate the interventional density of an observation x\T=a with FCause as:

$${p}_{θ} ( {x}_{\backslash T} = a \vert {x}_{T} = b , {G}_{do ( {x}_{T} )}^{m} ) = \frac{{p}_{θ} ( {x}_{\backslash T} = a \vert {x}_{T} = b , {G}_{do ( {x}_{T} )}^{m} ) {p}_{θ} ( {x}_{T} = b \vert {G}_{do ( {x}_{T} )}^{m}}{{p}_{θ} ( {x}_{T} = b \vert {G}_{do ( {x}_{T} )}^{m} )} = {\prod }_{j ∈ \backslash T} p ( {z}_{i} = ( {a}_{i} - {f}_{i} ( {a}_{Pa ( i ; {G}_{do ( {x}_{T} )}^{m} )} ) ) ) \qquad ( 17 )$$

It is then possible marginalise the graph using Monte Carlo as in Eq. (15). We can draw samples x^{m} from p_{θ}(x_{\T}|do(x_{T}=b)) as:

$${x}_{j}^{m} = f ( {x}_{Pa ( j ; {G}_{do ( {x}_{T} )}^{m} )}^{m} ) + {z}_{j}^{m} ; \qquad ( 18 )$$

$${G}^{m} \sim {q}_{ϕ} ( G ) ;$$

$${z}_{j}^{m} \sim \{ \begin{matrix}δ ( {z}_{j} - {b}_{j} ) , & if j ∈ T , \\ p ( z ) , & else\end{matrix} .$$

while noting that Pa(j;G_{do(x}_{_{T}}₎^{m})=Ø∀′, j∈T. One can use these to obtain a Monte Carlo estimate of the expectations required for ATE computation, as defined in Eq. (4).

CATE computation: Evaluating conditional densities

$${p}_{θ} ( {x}_{Y} \vert do ( {x}_{T} = b ) , {x}_{C} = c , G ) = \frac{{p}_{θ} ( {x}_{Y} , {x}_{T} = b , {x}_{C} = c , {G}_{do ( {x}_{T} )} )}{{p}_{θ} ( {x}_{T} = b , {G}_{do ( {x}_{T} )} ) {p}_{θ} ( {x}_{C} = c \vert {x}_{T} = b , {G}_{do ( {x}_{T} )} )} \qquad ( 19 )$$

is not necessarily straightforward for FCause-style models, due to the intractability of the conditional densities of the form p_{θ}(x_{C=c}|x_{T=b}, G_{do(x}_{_{T}}₎).

FCause's imputation inference network, introduced in Eq. (10), allows approximation of arbitrary conditionals q_{ψ}(x_{C}|x_{T}), but implicitly assumes a fixed graph. For the purpose of conditional treatment estimation, there is introduced herein a new graph aware inference model q_{ψ}(x_{C}|x_{T}, G). To introduce this graph-awareness, in embodiments first the functional form of the FCause is modified by introducing an additional edge network h: [symbol] → [symbol] parametrised by θₕ:

$${x}_{i} = {g}^{d} ( {\sum }_{j ∈ Pa ( i ; G )} {G}_{ji} h ( {g}^{e} ( {x}_{j} , j ; {θ}_{e} ) , j , i ) , i ; {θ}_{d} ) + {z}_{i} ; \qquad ( 20 )$$

$${z}_{i} \sim ( 0 , {σ}_{i}^{2} I )$$

which takes as inputs an embedding and the indices of the child node and parent mode. This model is meant to explicitly model the functions at the edges discussed earlier and provides a generalisation of the FCasuse SEM.

Now is described the form of a graph aware inference model according to certain embodiments disclosed herein. It shares the encoder and edge networks described earlier, allowing the successive application of multiple edge operators h(⋅,n,m) . . . ° h(⋅, i, k)° h(⋅,j,i)° gₑ(⋅,j) to simulate a traversal through the graph. Denoted by [symbol] (⋅,i,j,G;θₕ) is the composition of the edge network h along the nodes placed in topological order from i to j in graph G. The symbol θₕ is used to refer to the edge model's parameters. Also introduced is a variational decoder network g^{v}: [symbol] → [symbol] , with dᵥ referring to the size of the parameter space of q_{ψ}(x_{C}|x_{T}, G). Again the variational parameters are referred to as ψ. The variational decoder g^{v} replaces the standard FCause decoder, resulting in an inference distribution of the form:

$${q}_{ψ} ( {x}_{C} \vert {x}_{T} , G ) = q ( {x}_{C} ; {g}^{v} ( {\sum }_{j ∈ T} {h}_{} ( \cdot , j , i , {θ}_{h} ) ∘ {g}^{e} ( {x}_{j} , j ; {θ}_{e} ) , i ; ψ ) ) . \qquad ( 21 )$$

The new graph aware inference model acts as a plug-in replacement for the imputation model in Eq. (9) and can thus be trained with the same ELBO. By plugging the approximate distribution Eq. (21) into the interventional distribution Eq. (19), it is possible to approximate conditional densities.

The expectations over effect variables x_{ε}, involved in CATE computation, are approximated using Monte Carlo. Simulating from the described system will involve sampling from the variational approximation variables which are ancestors to both the effect variables and conditioning variables. This ancestor set within graph G may be denoted as An(E,C,G). Again marginalising over graphs, samples are drawn from x_{E}^{m} from p_{θ}(x_{\E}|do(x_{T=b}), x_{C=c}) as:

$${x}_{j}^{m} \{ \begin{matrix}\sim {q}_{ψ} ( {x}_{j} \vert {x}_{C} , {x}_{T} , {G}^{m} ) , & if j ∈ An ( E , C , G ) , \\ = f ( {x}_{Pa ( j ; {G}_{do ( {x}_{T} )}^{m} )}^{m} ) + {z}_{j}^{m} & else\end{matrix} ; \qquad ( 22 )$$

$$with :$$

$${G}^{m} \sim {q}_{ϕ} ( G ) ; \qquad ( 23 )$$

$${z}_{j} = \{ \begin{matrix}δ ( {z}_{j} - b ) , & if j ∈ T , \\ p ( z ) , & else\end{matrix} .$$

### FCause++

Further embodiments may further build upon the Fcause framework, including a number of additional improvements.

Utilizing domain knowledge: Whilst the described model is able to learn a posterior over graphs entirely from data, in many settings it is most useful to combine the ability to learn the causal structure from data with some limited prior knowledge provided by domain experts. In FCause, the user may be allowed to partially specify the graph, allowing unknowns in the graph to be inferred from data. For example, using the ENCO parametrization, the user may be allowed to make a prior assumption for each edge of the graph. For a pair of nodes i, j, one of the following assumptions may be specified:

1. the edge i→j exists,

2. the edge j→i exists,

3. no edge exists between i and j,

4. the undirected edge i-j exists, but its orientation

5. should be learnt from data,

6. an edge between i and j must be oriented i→j, but

7. its existence should be learnt from data,

8. an edge between i and j must be oriented j→i, but

9. its existence should be learnt from data,

10. the existence and orientation of an edge between i and

11. j should be learnt (default).

Given these fixed assumptions, a Bayesian posterior may then be learned over the unknown aspects of the graph, resulting in a Bayesian posterior over graphs that are consistent with the prior constraints. Whilst FCause is also compatible with probabilistic priors, embodiments focus on the constraint-based prior in certain implementations.

Non-Gaussian noise: a limitation of existing functional causal discovery methods is their assumption that the additive SEM noise is Gaussian. This assumption is sometimes made implicitly, through the choice of a squared norm training loss, or explicitly by optimising the parameters of a Gaussian density (cite FCause). In embodiments disclosed herein, this assumption may be relaxed, e.g. by incorporating a flow noise model:

$$p ( {z}_{i} ) = 𝒩 ( {ψ}^{- 1} ( {z}_{i} ) , 0 , 1 ) \vert \frac{\partial {ψ}^{- 1} ( {z}_{i} )}{\partial {z}_{i}} \vert , \qquad ( 24 )$$

where the learnable bijection ψ may be chosen to be a rational quadratic spline. The SEM may involve factorisation across our sets of exogenous variables p(z)=Qi p(zi). This does not allow coupling across sets of variables, disallowing it altogether for the 1d case where zᵢ∈R.

Handling mixed type data: In many applications, it is necessary to deal with data in which some nodes take continuous values, whilst others are discrete. To deal with this mixed-type data, the FCause may be recast without reference to the latent variables z. Given a graph G, and neural parameters θ, FCause provides a SEM density model for x of the form:

$${p}_{θ} ( x \vert G ) = {\prod }_{i = 1}^{D} p ( {x}_{i} \vert {x}_{{pa}_{G} ( i )} ) . \qquad ( 25 )$$

For continuous nodes, the form of the conditional density may be an additive model:

$${p}_{θ}^{cts} ( {x}_{i} \vert {x}_{{pa}_{G} ( i )} ) = {p}_{{ε}_{θ}^{( i )}} ( {x}_{i} - {μ}_{θ}^{( i )} ( {x}_{{pa}_{G} ( i )} ) ) \qquad ( 26 )$$

where ε_{θ}⁽ⁱ⁾ may be for example either a Gaussian with learned variance, or a spline flow. Written in this way, it is possible to extend FCause to support some xᵢ that are discrete. Rather than parametrizing the conditional density with an additive model, it is possible to parametrize the class probabilities directly:

$${p}_{θ}^{discrete} ( {x}_{i} \vert {x}_{{pa}_{G}} ( i ) ) = {P}_{θ}^{( i )} ( {x}_{{pa}_{G}} ( i ) ) ( {x}_{i} ) \qquad ( 27 )$$

where

$${P}_{θ}^{( i )} ( {x}_{{pa}_{G}} ( i ) )$$

is a normalised probability vector over the number of classes of xᵢ, and is a function of

$${x}_{{pa}_{G}} ( i ) .$$

This approach naturally provides an interventional density model for interventional data.

It will be appreciated that the above embodiments have been described by way of example only.

More generally, according to one aspect disclosed herein there is provided computer-implemented method comprising: accessing a machine learning, ML, model that is operable to sample a causal graph from a graph distribution describing different possible graphs, wherein nodes represent the different variables of said set and edges represent causation, and the graph distribution comprises a matrix of probabilities of existence and causal direction of potential edges between pairs of nodes, and wherein the ML model is trained to be able to generate a respective simulated value of a selected variable from among said set based on the sampled causal graph; wherein the method comprises using the ML model to estimate a treatment effect from one or more intervened-on variables on another, target variable from among the variables of said set, by: a) selecting the target variable as the selected variable to be simulated by the ML model; b) fixing the input value of each intervened-on variable to a specified value, including disregarding any edge directed from any parent of the intervened-on variable into the intervened-on variable in the sampled causal graph; c) sampling a causal graph from the graph distribution and observing the corresponding simulated value of the target variable; d) repeating c) multiple times, re-sampling the causal graph from the graph distribution each time; and e) determining an expectation of the target variable by averaging the simulated values of the target variable from c)-d) over the multiple sampled graphs, thus giving the estimated treatment effect.

In embodiments, the ML model may comprise a respective encoder and decoder for each respective one of a set of variables, each encoder being arranged to encode an input value of its respective variable into a respective embedding, and the ML model further comprising a selector, a combiner and a demultiplexer. In this case the selector is operable to: perform the sampling of a causal graph from the graph distribution; and perform the selecting of the selected variable from the sampled causal graph, identify which other of the variables are parents of the selected variable in the sampled causal graph, and input the embeddings of the identified parents into the combiner to produce a combined embedding, the demultiplexer being arranged to input the combined embedding into the respective decoder of the selected variable to generate a respective simulated value. In such embodiments, a) comprises operating the selector to select the target variable as the selected variable, and c) comprises operating the selector to perform the sampling of the sampled causal graph from the graph distribution, thereby causing the respective decoder of the target variable to generate the respective simulated value based on the embeddings of the parents of the target variable.

In embodiments, said averaging may comprise determining an average treatment effect, by: estimating a first expectation of a probabilistic distribution of the target variable given the specified value of each intervened-on variable; estimating a second expectation of a probabilistic distribution of the target variable without the specified value of at least one of the one or more intervened-on variables or with a different value of at least one of the one or more intervened-on variables; and determining a difference between the first and second expectations of the probabilistic distribution, thus giving the average treatment effect as the estimated treatment effect of the target variable.

In embodiments, the ML model may further comprises an inference network disposed between an unobserved one of the variables of said set and one or more observable ones of the variables of said set, arranged to infer the unobserved variable from the one or more observables variables. In such embodiments, the average treatment effect being estimated may comprise a conditional average treatment effect. In this case, the first expectation comprises an expectation of a probabilistic distribution of the target variable given the specified value of each intervened-on variable conditional on the input value of at least one of the observed variables other than the intervened-on variable; and the second estimation comprises an expectation of a probabilistic distribution of the target variable without the specified value of at least one of the one or more intervened-on variables, or with a different value of at least one of the intervened-on variables, but still conditional on the input value of said at least one observed variable.

In embodiments, the one or more intervened-on variables may model a treatment on a real-world entity and the target variable may model an effect of the treatment applied to the real-world entity. The method may further comprise actioning the treatment on the real-world entity in dependence on the estimated treatment effect.

For example, the real-world entity may comprise a living being, in which case the effect may comprise a symptom of the living being, and the treatment may comprise a medical treatment to the living being. As another example, the real-world entity may comprise a mechanical, electrical or electronic device or system, or an item of software; in which case the effect may comprise a state of the device, system or software; and the treatment may comprise an act of maintaining, debugging, upgrading or controlling the device, system or software.

In embodiments, the generating of the simulated value of the selected variable may comprise generating a noiseless value of the selected variable, sampling a random noise value, and combining the noiseless value with the sampled noise value to produce the simulated value.

In embodiments the combined embedding may comprise a sum of the embeddings of the identified parents.

In embodiments each of the encoders and the decoders may comprise a neural network.

In embodiments, the method may comprise training the ML model prior to the determination of the estimated treatment effect. Alternatively the ML model may have been pre-trained by another party.

The training may comprise, for each of a plurality of input data points in a training data set, each data point comprising a different set of input values for the set of input variables:
operating the selector to sample a causal graph from the graph distribution, and
for each respective one of the variables in said set, operating the selector to set the variable as the selected variable so as to generate a respective reconstructed value of the respective variable, the reconstructed values for the input data point thus forming a corresponding reconstructed data point; and

over the plurality of data points, simultaneously training the probabilities in the graph distribution along with the encoders and decoders so as to minimize a measure of difference between the input data points and the reconstructed data points.

In embodiments the measure of difference may comprise an ELBO function.

In embodiments, the inference network may be trained simultaneously with the encoders, decoders and graph distribution. Or as another, alternative example, the inference network may be trained in a subsequent training phase after said training of the encoders, decoders and graph distribution. In the latter case, the training may be performed in response to a query which specifies the input value of each intervened-on variable and requests to make the estimation, wherein the subsequent training phase may be performed by: simulating data from a sub-model comprising the ML model without the inference network while applying the input value specified in the query, and estimating the conditional average treatment effect by inputting the value of the observed variable into the inference network to return a distribution over effects from which the estimated treatment effect is computed.

In embodiments, the inference network may comprise a neural network.

In embodiments, the machine learning model may be hosted on a server system of a first party, the server system comprising one or more server units at one or more sites. In this case the method may further comprise, by the server system of the first party: providing an application programming interface, API, enabling a second party to contact the server system via a network; receiving a request from the second party over the network via the API; in response to the request, determining the estimated treatment effect on the target variable; and returning the estimated treatment effect to the second party over the network via the API.

According to another aspect disclosed herein, there is provided a computer program embodied on non-transitory computer-readable storage and configured so as when run on one or more processors to perform any of the methods disclosed herein.

According to another aspect there is provided a system comprising: processing apparatus comprising one or more processors; and memory comprising one or more memory units, wherein the memory stores code arranged to run on the processing apparatus and being configured so as when run to perform any of the methods disclosed herein.

Other variants or use cases of the disclosed techniques may become apparent to the person skilled in the art once given the disclosure herein. The scope of the disclosure is not limited by the described embodiments but only by the accompanying claims.

1. A computer-implemented method comprising:
accessing a machine learning, ML, model that is operable to sample a causal graph from a graph distribution describing different possible graphs, wherein nodes represent the different variables of said set and edges represent causation, and the graph distribution comprises a matrix of probabilities of existence and causal direction of potential edges between pairs of nodes, and wherein the ML model is trained to be able to generate a respective simulated value of a selected variable from among said set based on the sampled causal graph;
wherein the method comprises using the ML model to estimate a treatment effect from one or more intervened-on variables on another, target variable from among the variables of said set, by:
a) selecting the target variable as the selected variable to be simulated by the ML model;
b) fixing the input value of each intervened-on variable to a specified value, including disregarding any edge directed from any parent of the intervened-on variable into the intervened-on variable in the sampled causal graph;
c) sampling a causal graph from the graph distribution and observing the corresponding simulated value of the target variable;
d) repeating c) multiple times, re-sampling the causal graph from the graph distribution each time; and
e) determining an expectation of the target variable by averaging the simulated values of the target variable from c)-d) over the multiple sampled graphs, thus giving the estimated treatment effect.

2. The method of claim 1, wherein the ML model comprises a respective encoder and decoder for each respective one of a set of variables, each encoder being arranged to encode an input value of its respective variable into a respective embedding, and the ML model further comprising a selector, a combiner and a demultiplexer; wherein the selector is operable to:
perform the sampling of a causal graph from the graph distribution; and
perform the selecting of the selected variable from the sampled causal graph, identify which other of the variables are parents of the selected variable in the sampled causal graph, and input the embeddings of the identified parents into the combiner to produce a combined embedding, the demultiplexer being arranged to input the combined embedding into the respective decoder of the selected variable to generate a respective simulated value; and
wherein a) comprises operating the selector to select the target variable as the selected variable, and c) comprises operating the selector to perform the sampling of the sampled causal graph from the graph distribution, thereby causing the respective decoder of the target variable to generate the respective simulated value based on the embeddings of the parents of the target variable.

3. The method of claim 2, wherein the combined embedding comprises a sum of the embeddings of the identified parents.

4. The method of claim 2, wherein each of the encoders and the decoders comprises a neural network.

5. The method of claim 2, comprising training the ML model prior to the determination of the estimated treatment effect, the training comprising, for each of a plurality of input data points in a training data set, each data point comprising a different set of input values for the set of input variables:
operating the selector to sample a causal graph from the graph distribution, and
for each respective one of the variables in said set, operating the selector to set the variable as the selected variable so as to generate a respective reconstructed value of the respective variable, the reconstructed values for the input data point thus forming a corresponding reconstructed data point; and
over the plurality of data points, simultaneously training the probabilities in the graph distribution along with the encoders and decoders so as to minimize a measure of difference between the input data points and the reconstructed data points.

6. The method of claim 5, wherein the measure of difference is an ELBO function.

7. The method of claim 5, wherein said averaging comprises determining an average treatment effect, by:
estimating a first expectation of a probabilistic distribution of the target variable given the specified value of each intervened-on variable;
estimating a second expectation of a probabilistic distribution of the target variable without the specified value of at least one of the one or more intervened-on variables or with a different value of at least one of the one or more intervened-on variables; and
determining a difference between the first and second expectations of the probabilistic distribution, thus giving the average treatment effect as the estimated treatment effect of the target variable;
wherein the ML model further comprises an inference network disposed between an unobserved one of the variables of said set and one or more observable ones of the variables of said set, arranged to infer the unobserved variable from the one or more observables variables; wherein the average treatment effect being estimated comprises a conditional average treatment effect;
wherein the first expectation comprises an expectation of a probabilistic distribution of the target variable given the specified value of each intervened-on variable conditional on the input value of at least one of the observed variables other than the intervened-on variable;
wherein the second estimation comprises an expectation of a probabilistic distribution of the target variable without the specified value of at least one of the one or more intervened-on variables, or with a different value of at least one of the intervened-on variables, but still conditional on the input value of said at least one observed variable; and
wherein one of:
I) the inference network is trained simultaneously with the encoders, decoders and graph distribution; or
II) the inference network is trained in a subsequent training phase after said training of the encoders, decoders and graph distribution, in response to a query which specifies the input value of each intervened-on variable and requests to make the estimation, wherein the subsequent training phase is performed by: simulating data from a sub-model comprising the ML model without the inference network while applying the input value specified in the query, and estimating the conditional average treatment effect by inputting the value of the observed variable into the inference network to return a distribution over effects from which the estimated treatment effect is computed.

8. The method of claim 1, wherein said averaging comprises determining an average treatment effect, by:
estimating a first expectation of a probabilistic distribution of the target variable given the specified value of each intervened-on variable;
estimating a second expectation of a probabilistic distribution of the target variable without the specified value of at least one of the one or more intervened-on variables or with a different value of at least one of the one or more intervened-on variables; and
determining a difference between the first and second expectations of the probabilistic distribution, thus giving the average treatment effect as the estimated treatment effect of the target variable.

9. The method of claim 8, wherein the ML model further comprises an inference network disposed between an unobserved one of the variables of said set and one or more observable ones of the variables of said set, arranged to infer the unobserved variable from the one or more observables variables; wherein the average treatment effect being estimated comprises a conditional average treatment effect, and wherein:
the first expectation comprises an expectation of a probabilistic distribution of the target variable given the specified value of each intervened-on variable conditional on the input value of at least one of the observed variables other than the intervened-on variable;
the second estimation comprises an expectation of a probabilistic distribution of the target variable without the specified value of at least one of the one or more intervened-on variables, or with a different value of at least one of the intervened-on variables, but still conditional on the input value of said at least one observed variable.

10. The method of claim 9, wherein the inference network comprises a neural network.

11. The method of claim 1, wherein the one or more intervened-on variables model a treatment on a real-world entity and the target variable models an effect of the treatment applied to the real-world entity, and the method further comprises actioning the treatment on the real-world entity in dependence on the estimated treatment effect.

12. The method of claim 11, wherein one of:
the real-world entity comprises a living being, the effect comprises a symptom of the living being, the treatment comprises a medical treatment to the living being; or
the real-world entity comprises a mechanical, electrical or electronic device or system or an item of software, the effect comprises a state of the device, system or software, and the treatment comprises an act of maintaining, debugging, upgrading or controlling the device, system or software.

13. The method of claim 1, wherein the generating of the simulated value of the selected variable comprises generating a noiseless value of the selected variable, sampling a random noise value, and combining the noiseless value with the sampled noise value to produce the simulated value.

14. The method of claim 1, wherein the machine learning model is hosted on a server system of a first party, the server system comprising one or more server units at one or more sites; and the method further comprises, by the server system of the first party:
providing an application programming interface, API, enabling a second party to contact the server system via a network;
receiving a request from the second party over the network via the API;
in response to the request, determining the estimated treatment effect on the target variable; and
returning the estimated treatment effect to the second party over the network via the API.

15. A computer program embodied on non-transitory computer-readable storage and configured so as when run on one or more processors to perform a method comprising:
accessing a machine learning, ML, model that is operable to sample a causal graph from a graph distribution describing different possible graphs, wherein nodes represent the different variables of said set and edges represent causation, and the graph distribution comprises a matrix of probabilities of existence and causal direction of potential edges between pairs of nodes, and wherein the ML model is trained to be able to generate a respective simulated value of a selected variable from among said set based on the sampled causal graph;
wherein the method comprises using the ML model to estimate a treatment effect from one or more intervened-on variables on another, target variable from among the variables of said set, by:
a) selecting the target variable as the selected variable to be simulated by the ML model;
b) fixing the input value of each intervened-on variable to a specified value, including disregarding any edge directed from any parent of the intervened-on variable into the intervened-on variable in the sampled causal graph;
c) sampling a causal graph from the graph distribution and observing the corresponding simulated value of the target variable;
d) repeating c) multiple times, re-sampling the causal graph from the graph distribution each time; and
e) determining an expectation of the target variable by averaging the simulated values of the target variable from c)-d) over the multiple sampled graphs, thus giving the estimated treatment effect.

16. A system comprising:
processing apparatus comprising one or more processors; and
memory comprising one or more memory units, wherein the memory stores code arranged to run on the processing apparatus and being configured so as when run to perform a method comprising:
accessing a machine learning, ML, model that is operable to sample a causal graph from a graph distribution describing different possible graphs, wherein nodes represent the different variables of said set and edges represent causation, and the graph distribution comprises a matrix of probabilities of existence and causal direction of potential edges between pairs of nodes, and wherein the ML model is trained to be able to generate a respective simulated value of a selected variable from among said set based on the sampled causal graph;
wherein the method comprises using the ML model to estimate a treatment effect from one or more intervened-on variables on another, target variable from among the variables of said set, by:
a) selecting the target variable as the selected variable to be simulated by the ML model;
b) fixing the input value of each intervened-on variable to a specified value, including disregarding any edge directed from any parent of the intervened-on variable into the intervened-on variable in the sampled causal graph;
c) sampling a causal graph from the graph distribution and observing the corresponding simulated value of the target variable;
d) repeating c) multiple times, re-sampling the causal graph from the graph distribution each time; and
e) determining an expectation of the target variable by averaging the simulated values of the target variable from c)-d) over the multiple sampled graphs, thus giving the estimated treatment effect.
